(19) United States
(12) Patent Application Publication
(10) Pub. No.: US 2011/0076647 A1
(43) Pub. Date: Mar. 31, 2011

(54) DENTAL ELEMENT CODING

(75) Inventors: Thomas DITZEL, Nidderau (DE); Peter NEUMEIER, Muenchen (DE)

(73) Assignee: DITZEL GMBH & CO. KG GRUNDSTUECKS-UND BETEILIGUNGSGESELLSCHAFT, Schoeneck (DE)

(21) Appl. No.: 12/888,942

(22) Filed: Sep. 23, 2010

(30) Foreign Application Priority Data
Sep. 29, 2009 (DE) 10 2009 043 281.7

(51) Int. Cl. A61B 5/117

(52) U.S. Cl. 433/229

(57) ABSTRACT

Dental identifier and coding method for a human or animal. The dental identifier includes a coding arranged on at least a portion of a dental element, while the method includes applying a coding to a dental element.

### CROSS-REFERENCE TO RELATED APPLICATIONS

The present application claims priority under 35 U.S.C. §119 of German Patent Application No. 10 2009 043 281.7, filed on Sep. 29, 2009, the disclosure of which is expressly incorporated by reference herein in its entirety.

### BACKGROUND OF THE INVENTION

1. Field of the Invention

The invention relates to a dental element, which can be, at least in part, an artificial dental element.

Furthermore, the invention relates to a method for applying a coding.

2. Discussion of Background Information

From the review presentation “Identifikation unbekannter Toter durch odontologische Untersuchungen” [Identification of unknown dead bodies by means of odontological examinations] (Zeitschrift Rechtsmedizin, Vol. 11, p. 37-41, 2001; Springer Berlin Heidelberg) a method is known for identifying unknown dead bodies based on examinations of the dental system. A method of this type is carried out in particular when other identifying features (such as personal papers or fingerprints) are not available to a sufficient extent. Teeth exhibit dental features with the aid of which people can be positively identified. Furthermore, teeth are extremely tough. Tooth enamel is considered to be the hardest substance of the human body. The fact that teeth are registered and documented in the scope of dental treatments in civilized regions can be utilized in the identification. With a complete documentation, the identification can be carried out based on premortal and postmortem findings. Alternatively, a comparison of premortal and postmortem x-ray images can lead to identification. In particular, orthopantomograms make it possible for the coroner to make a reliable identification.

Although methods of this type have proven useful in principle, in particular, in cases of burns, where teeth often represent the only remaining option for identification, an identification of this type requires a considerable amount of documentation for the treating dentist. This documentation is also made difficult by the fact that many patients often change their dentist, for example, due to relocation. X-ray images are accepted by sectors of the population only in the case of absolute necessity because of the radiation exposure.

### SUMMARY OF THE INVENTION

Embodiments of the invention provide a simple method for identification of a person or an animal.

According to embodiments, a dental element of the type mentioned at the outset is provided with a coding that is arranged on the dental element.

With this embodiment, information can be attached to the dental element in a simple manner. Reading out a coding of this type is much easier and quicker compared to an examination of the dental system. The attending dentist or dentists no longer need to be found; instead the coding is simply read out on the spot. This does not entail any radiation exposure, either. In this manner medical measures can be taken quickly and/or family members can be notified. Reading out the coding is carried out via a reader device such as a scanner or a camera.

Teeth or parts of the jawbone of the person or animal are conceivable as a dental element. However, it is also possible to place the coding on the artificial dental element. In particular, it is possible to attach the coding to a filling, a bridge, an inlet, an implant, a denture or quite generally to a dental prosthesis part. The arrangement of the coding on the artificial dental element is thereby particularly advantageous. It can be attached outside a body of the person or the animal. There is therefore no need to take the body into consideration in the attachment.

In particular, an individual person-related or animal-related coding is conceivable. This can contain information on the place of birth, place of residence, name, blood group, illnesses, required medicine or other biometric data on the person or animal. The person to be contacted in the event of an accident can also be stored.

Preferably, the coding is embodied or formed, e.g., as a color application, mark or marker. This color application can have an individual person-related coloring. The coloring can be embodied or formed such that the coloring can be unambiguously assigned to the person to be identified, similar to a fingerprint. The color application can be arranged on the dental element in an insoluble manner.

Preferably, the coding has, e.g., an optoelectronically readable lettering. The optoelectronically readable lettering can be, e.g., a bar code. The information can therefore be stored in binary symbols. One-dimensional or multidimensional codings are conceivable.

Preferably, the coding may be embodied or formed, e.g., as a nanostructure. The coding can be so small that on the one hand it is not visible to the human eye. On the other hand, however, it is easily possible for the reader device to read out the coding. The person bearing the coded dental element therefore does not suffer any aesthetic disadvantages. The danger of information contained in the coding being given to unauthorized persons is thus also reduced.

Preferably, a coding element of the coding can be read in a predetermined wavelength interval. The wavelength interval can be in an invisible portion of the light. This has the advantage that unauthorized persons cannot read the coding without technical equipment.

Preferably, coding elements of the coding are respectively readable in predetermined wavelength intervals differing from one another. Part of the coding can therefore be contained in a specific wavelength interval, while another part of the coding is part of a different wavelength interval. The wavelength intervals can thereby overlap, but they can also be disjointed. The information contained in the respective coding elements can then be read and thereafter combined with optical filters and wavelength-sensitive receiver elements. A data processing device can be provided for this purpose. In this manner, a high information density is also obtained, since several wavelength intervals, i.e., channels, are used simultaneously.

The coding can preferably be decoded only with a decoder. Unauthorized persons thus cannot read the coding. The decoder can also have an additional data processing device. The decoder then uses the information of the coding and decodes it with the aid of the data processing device.

Preferably, the coding can have a heat protectant. In particular in the case of burns, this ensures that the coding remains readable and undamaged. The heat protectant can also be embodied or formed as a coating. However, it is also possible for a mixture to be added to the coding, which mixture makes the coding heat-resistant.

According to embodiments, a method of the type mentioned at the outset includes applying a coding to a dental element, which can be at least in part an artificial dental element.

In this case, it is no longer necessary to identify the person or the animal based on odontological examinations. Once the coding has been applied, information stored in the coding can be easily read out. The coding can contain biometric information, but also any other information, as already mentioned. Moreover, such biometric and/or other information can be stored in a database and can be retrieved upon reading the coding or data contained in the coding. Parts of human or animal teeth can be used as a dental element. However, artificially produced dental elements can also be used.

Preferably, the coding can be applied, e.g., by a color application, mark or marker. The application can be carried out, for example, with a device such as is known from patent DE 10 2006 061 893. The color application can be applied such that it can be unambiguously assigned to the person or the animal.

Preferably, the coding may be provided with a heat protectant. The heat protectant can be applied after the coding has been applied. However, this can also take place in one step, in that a heat-resistant mixture is added to the coding.

Preferably, the coding can be encoded. Using one or more codes, readable information may be converted into secret information. Particularly sensitive biometric data can then only be read by an authorized person in possession of the code.

It is advantageous if different predetermined wavelength intervals are used to encode the coding and/or if certain parts of the information stored in the coding are readable only in certain wavelength intervals. In order to be able to read the coding, different optical filters and/or wavelength-sensitive optoelectrical receiver elements are used. With the aid of a data processing device, the read out information can then be assembled and the complete information stored in the coding is thus obtained.

Preferably, the coding is read out and the information contained in the coding is compared with further information. The information contained in the coding, which is contained, for example, in the color application, can be read out first. The information thus obtained is then compared to further information, which is stored, for example, in a data file. The person bearing the dental element can thus be identified and additional information about the person can possibly be obtained.

Embodiments of the invention are directed to a dental identifier. The dental identifier includes a coding arranged on at least a portion of a dental element.

According to embodiments, the dental element may include, at least in part, an artificial dental element.

In accordance with embodiments, the coding may include a color marking.

According to other embodiments, the coding can include optoelectronically readable lettering.

According to still other embodiments, the coding may include a nanostructure.

Moreover, in embodiments, the coding can include at least one coding element readable in a predetermined wavelength interval. Further, the at least one coding element may include a plurality of coding elements that are respectively readable in predetermined wavelength intervals differing from one another.

According to still further embodiments, the coding can be decodable with a decoder.

In accordance with further embodiments, a heat protectant may be formed on at least one of the coding and the at least a portion of the dental element. The heat protectant can at least one of be part of the coding or surround the coding.

Embodiments of invention are directed to a method for coding. The method includes applying a coding to a dental element.

In accordance with embodiments, the dental element may include at least in part an artificial dental element.

According to other embodiments, the coding can be applied by application of a color mark.

In accordance with still other embodiments, the coding can at least one of include a heat protectant and be covered by a heat protectant.

According to still other embodiments of the invention, the coding may be encoded. Further, different predetermined wavelength intervals can be used to encode the coding. Moreover, the method can include reading encoded information contained in the coding, decoding the encoded information, and comparing the decoded information to stored information.

According to other embodiments, the method can include reading information contained in the coding, and comparing the information to stored information.

In accordance with still yet other embodiments of the present invention, the coding can be applied to at least one of a natural tooth and an artificial dental element.

Embodiments of the invention are directed to an identification system. The system includes a coding applied to at least one of a natural tooth and an artificial dental element of a human or animal, a reading device structured and arranged to read information contained in the coding, and a comparing unit structured and arranged to compare the information read from the coding to stored information in a database. The database further contains data regarding the identity of the human or animal that is associated with the stored information.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 illustrates a dental element embodied as a tooth with several codings, in which a part of the tooth is replaced by an artificial dental element;

FIG. 2 illustrates a sectional view through a crown having a coding and a heat protectant;

FIG. 3 diagrammatically illustrate an information density as a function of a wavelength; and

FIG. 4 diagrammatically illustrates an arrangement of a reader device, a decoder, a data processing device, and a data file.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

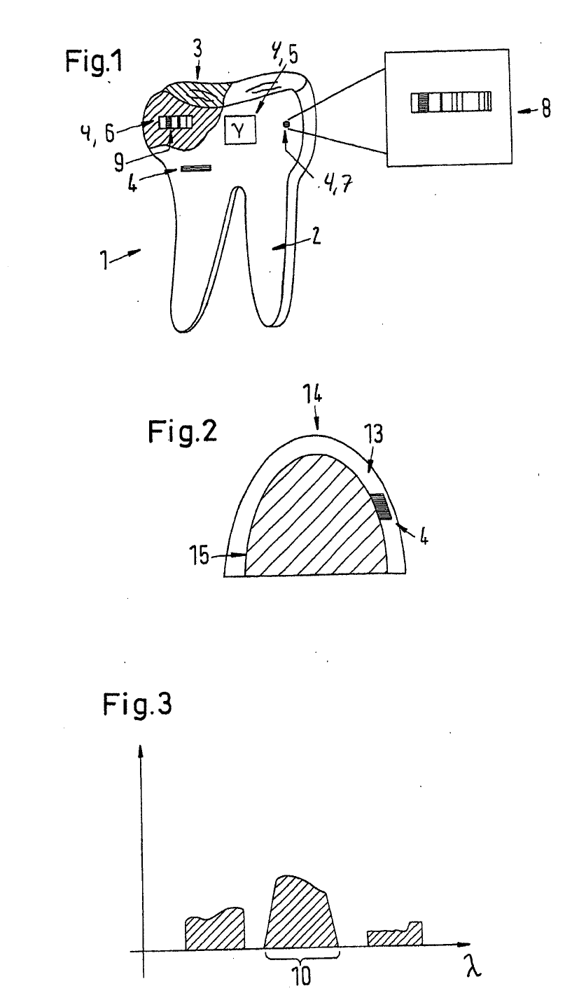

FIG. 1 shows a dental element 1 embodied as a tooth. The dental element 1 can include a natural dental element 2 and/or an artificial dental element 3 in various combinations. One or more codings or codes 4 may be arranged on dental element 1. Codings 4 can be arranged on natural dental element 2 and/or on artificial dental element 3 or even on a combination of dental elements 2 and 3. Natural dental element 2 can be a tooth or a part of the jawbone of a human (or animal) set of teeth. Artificial dental element 3 can be embodied or formed as a filling, bridge, inlet, implant, denture or as a dental prosthesis part.

Biometric information about a person or an animal can be stored in the coding and/or can be stored in a database and retrieved by the coding or information contained in the coding.

Coding 4 can be embodied or formed with different designs. In particular, it is conceivable that coding 4 may be embodied or formed, e.g., as a color application (mark or marker) 5. Color application 5 can be permanently applied to dental element 1. In particular, a specific color of color application 5 can be selected from colors lying in the invisible range of light, which would make it difficult for an unauthorized person to read the coding 4.

Coding 4 can also be, e.g., an optoelectronically readable lettering 6. Optoelectronically readable lettering 6 can be embodied or formed as, e.g., a barcode. Moreover, coding 4 can be one-dimensional or multidimensional. It is possible to use the coding 4 as it is used in the retail trade. It is possible per se to embody or form coding 4 on dental element 1 such that it can be seen with the naked eye. However, in practice, coding 4 will be embodied or formed on dental element 1 so that it cannot be read or even seen with the naked eye. In particular embodiments, a nanostructure 7 can also be utilized for embodying or forming coding 4. Nanostructure 7 is shown in FIG. 1 on dental element 1, as shown in the enlarged section 8, can correspond to the coding by optoelectronically readable lettering 6, color application 5, or any other coding methods known to those ordinarily skilled in the art. In this manner, coding 4 is not noticeable to a third party, but would nevertheless still be readable.

Coding 4 can be embodied or formed by one or more coding elements 9, which can be readable in a predetermined wavelength interval 10 (see FIG. 3). Further, if the invisible part of light is selected as wavelength interval 10, then coding 4 would not visible, regardless of its size.

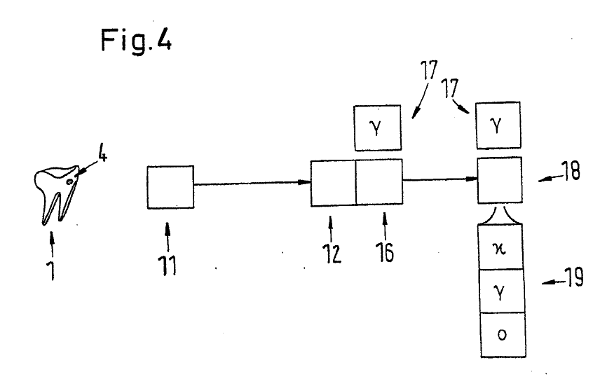

As shown in FIG. 3, coding elements 9 of coding 4 can also be embodied or formed as readable in predetermined wavelength intervals 10 that differ from one another. In FIG. 3, information density is plotted on the y-axis and a wavelength (λ) is plotted on the x-axis. For example, in wavelength interval 10, the information density is different from zero, i.e., at least one of the coding elements 9 is readable in this wavelength interval 10. Further, one or more wavelength intervals 10 can be provided, such that multiple wavelength intervals 10 can be utilized in a disjointed and/or in an overlapping manner. In order to be able to read out the entire coding 4, a reader device 11 (see FIG. 4) can be provided, which can include an optical filter and/or a wavelength-sensitive receiver element.

In order to be sure that no unauthorized person can read coding 4, it may be advantageous to encode coding 4 on dental element 1. In order to decode coding 4, a decoder 12 can be used, and, without which, coding 4 is preferably not readable. Moreover, different predetermined wavelength intervals 10 can also be used for encoding coding 4.

As shown in FIG. 2, a heat protectant 13 can also be provided. Heat protectant 13 prevents coding 4 from being damaged in fires, etc. Particularly with fires, in which identification documents, etc. may be burned, coding 4 remains in tact and readable. A crown 14, as a non-limiting example of dental element 1, is shown with coding 4 arranged on its surface 15. Heat protectant 13 can surround crown 14 and coding 4 but still allow coding 4 to be readable by a suitable reader, as discussed below. Heat protectant 13 can also take on a mechanical function, i.e., it can be made of hard material and thus prevent damage to crown 14 on which coding 4 is provided in the normal course of life, as well as in the event of an explosion. It is further noted that heat treatment 13 can be applied either at the time of the application of coding 4 or subsequently to the application of coding 4.

FIG. 4 shows dental element 1 with coding 4. Coding 4, which can be encoded, can be read out by reader device 11 in a conventional manner. When encoded, a decoder 12 can be arranged behind reader device 11 to decode coding 4 in a manner understood by those ordinarily skilled in the art. A data processing device 16 of conventional may be arranged behind decoder 12 in order to compare decoded information 17 contained in coding 4 with data or information 19 stored in a data file 18, in a manner understood by those ordinarily skilled in the art. Thus, for example, the person (or animal) can be identified based on a comparison of the read coding 4 and the data or information 19 contained in data file 18. However, it is further understood that data processing device 16 can also be utilized without decoder 12 and can also be utilized without data file 18, such that, e.g., the data is read directly from coding 4. Moreover, it should also be understood that coding 4 can be read through reading several individual coding elements 9 and combining these individual readings to form an entire coding 4 that contains information retrievable by data processing device 16.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A dental identifier comprising:
a coding arranged on at least a portion of a dental element.

2. The dental identifier in accordance with claim 1, wherein the dental element comprises, at least in part, an artificial dental element.

3. The dental identifier in accordance with claim 1, wherein the coding comprises a color marking.

4. The dental identifier in accordance with claim 1, wherein the coding comprises optoelectronically readable lettering.

5. The dental identifier in accordance with claim 1, wherein the coding comprises a nanostructure.

6. The dental identifier in accordance with claim 1, wherein the coding comprises at least one coding element readable in a predetermined wavelength interval.

7. The dental identifier in accordance with claim 1, wherein the at least one coding element comprises a plurality of coding elements that are respectively readable in predetermined wavelength intervals differing from one another.

8. The dental identifier in accordance with claim 1, wherein the coding is decodable with a decoder.

9. The dental identifier in accordance with claim 1, further comprising a heat protectant formed on at least one of the coding and the at least a portion of the dental element.

10. The dental identifier in accordance with claim 9, wherein the heat protectant at least one of is part of the coding or surrounds the coding.

11. A method for coding, comprising:
applying a coding to a dental element.

12. The method in accordance with claim 11, wherein the dental element comprises at least in part an artificial dental element.

13. The method in accordance with claim 11, wherein the coding is applied by application of a color mark.

14. The method in accordance with claim 11, wherein the coding at least one of comprises a heat protectant and is covered by a heat protectant.

15. The method in accordance with claim 11, wherein the coding is encoded.

16. The method in accordance with claim 15, wherein different predetermined wavelength intervals are used to encode the coding.

17. The method in accordance with claim 15, further comprising:
reading encoded information contained in the coding;
decoding the encoded information; and
comparing the decoded information to stored information.

18. The method in accordance with claim 11, further comprising:
reading information contained in the coding; and
comparing the information to stored information.

19. The method in accordance with claim 11, wherein the coding is applied to at least one of a natural tooth and an artificial dental element.

20. An identification system, comprising:
a coding applied to at least one of a natural tooth and an artificial dental element of a human or animal;
a reading device structured and arranged to read information contained in the coding; and
a comparing unit structured and arranged to compare the information read from the coding to stored information in a database,
wherein the database further contains data regarding the identity of the human or animal that is associated with the stored information.
